(19) United States
(12) Patent Application Publication
(10) Pub. No.: US 2004/0059413 A1
(43) Pub. Date: Mar. 25, 2004

(54) Suture template for facilitating implantation of a prosthetic heart valve

(76) Inventor: Claudio Argento, Long Beach, CA (US)

Correspondence Address: EDWARDS LIFESCIENCES CORPORATION, ONE EDWARDS WAY, IRVINE, CA, 92614, US

(21) Appl. No.: 10/663,006

(22) Filed: Sep. 16, 2003

Related U.S. Application Data
(60) Provisional application No. 60/412,415, filed on Sep. 20, 2002.

(51) Int. Cl. A61F 2/24

(52) U.S. Cl. 623/2.11; 623/2.41

(57) ABSTRACT

A suture template for facilitating implantation of a prosthetic valve includes a plurality of commissure portions connected to a plurality of cusp portions around an annulus. Each commissure portion has at least one notch and each cusp portion can have an inwardly directed ledge and at least one notch. In one embodiment, the notches open towards the bottom of the suture template. In another embodiment, the notches open towards the top of the suture template.

### RELATED APPLICATIONS

The present application claims priority to U.S. Provisional Application Serial No. 60/412,415, filed Sep. 20, 2002.

### FIELD OF THE INVENTION

The present invention is related generally to a suture template for assisting surgeons in placing sutures or marking locations where a valve prosthesis is to be attached.

### BACKGROUND

Prostheses are artificial devices used to repair or replace damaged or diseased organs, tissues and other structures in humans and animals. Prostheses must be generally bio-compatible since they are typically implanted for extended periods of time. For example, prostheses can include artificial hearts, artificial heart valves, ligament repair material, vessel repair material, surgical patches constructed of mammalian tissue and the like.

Prosthetic heart valves are used to replace diseased natural heart valves in the aortic, mitral, tricuspid and pulmonary positions in the heart. Examples of three such valves are shown in Carpentier et al. U.S. Pat. No. 4,106,129, Ionescu et al. U.S. Pat. No. 4,084,268 and Davis et al U.S. Pat. No. 4,192,020. As shown by these patents, a prosthetic heart valve typically includes a stent formed of a wire or a shell, and valve leaflets attached to the stent. U.S. Patent No. 4,501,030, issued to Lane, discloses another prosthetic heart valve which includes a frame having a plurality of commissure supports, a plurality of resilient supports, and a plurality of valve leaflets. The valve leaflets are attached to the resilient supports, and the resilient supports lie radially outwardly of the commissure supports, respectively. When in use, the valve is subjected to forces which are used to clamp the valve leaflets between the resilient supports and the commissure supports to augment any leaflet attachment techniques that may be used.

The natural aortic heart valve has three leaflets that open to allow flow into the aorta and close to prevent back flow into the left ventricle. Each of the three leaflets of the natural aortic heart valve is attached to the cylindrical wall of the aorta along a non-planar curve. A typical aortic prosthetic valve includes three valve leaflets attached to a post frame. Some relatively recent valve designs require that the valve be secured in position via an undulating suture line that generally follows the cusps and commissure supports of the wireframe. Coronary arteries, however, join the aorta near the valve. Thus the commissure post of the prosthetic heart valve, if located improperly in the aorta, can block or partially block a coronary artery. This complicates the placement of the prosthesis.

Valve replacement is typically performed during open-heart surgery. The natural valve is mounted in an annulus comprising dense fibrous rings attached either directly or indirectly to the atrial and ventricular muscle fibers. In a valve replacement operation, the damaged leaflets are typically excised and the annulus sculpted to receive the replacement valve. Ideally the annulus presents relatively healthy tissue which can be formed by the surgeon into a uniform ledge projecting into the orifice left by the removed valve. The time and spatial constraints imposed by surgery, however, often dictate that the shape of the resulting annulus is less than perfect for attachment of a sewing ring of the replacement valve. Moreover, the annulus may be calcified as well as the leaflets and complete annular debridement, or removal of the hardened tissue, results in a larger orifice and less defined annulus ledge to which to attach the sewing ring of the prosthesis. In short, the contours of the resulting annulus vary widely after the natural valve has been excised.

During replacement, the annulus is sized with an annulus sizer to determine the proper size of the artificial valve. The artificial valve is then positioned in the opening and the sewing ring is carefully sutured or sewn to the tissue surrounding the valve opening. The annulus sizer is typically cylindrical, and made of plastic with a central threaded tap to which a handle is attached. A number of sizers are at a surgeon's disposal, each having a different size, or diameter. In use the surgeon inserts the sizer into the valve opening, measuring the size of the opening. An artificial valve properly sized for the valve opening is then selected and sewn in place.

Prior to attaching the prosthetic valve to the annulus and/or aorta, it is also helpful for the surgeon to mark the location within the aorta where the prosthetic valve is to be attached. Failure to mark the proper location where the prosthetic valve is to be precisely attached could lead to undesirable consequences including improper placement of the prosthetic valve.

### SUMMARY

In one embodiment, a suture template for facilitating implantation of a prosthetic heart valve in a patient is disclosed. The suture template includes an annular body having a plurality of commissure portions and a plurality of cusp portions. The plurality of commissure portions are connected with each other utilizing the plurality of cusp portions. In one embodiment, each commissure portion of the suture template includes a pair of upstanding arms extending from the cusp portions, the arms coming together to form a tip and defining an elongated downwardly opening notch between the arms. The cusp portions can also be provided with at least one notch. The notches, which can open toward the top and/or bottom, facilitate the removal of the suture template. All notches on the commissure portions as well as cusp portions represent major reference points for suture placement for implanting the prosthetic valve.

In a further embodiment of the invention, a method of marking a location to implant a prosthetic heart valve in a patient, utilizing a suture template, is disclosed. The method includes placing the suture template in the location of the heart that is to receive the valve, placing a plurality of spaced apart sutures through notches along one end of the suture template and through and along the desired location of the heart and removing the suture template, and placing the sutures into the prosthetic valve that is to be implanted. Instead of a notch, any other physical or visual guide of the template may be used to assist the surgeon in suture placement.

In yet another embodiment, the invention provides a method of marking a location for implanting an artificial device by a surgeon. The method includes lowering a marking tool within a body cavity, positioning the marking tool where the artificial device is to be implanted within the body cavity, and triggering a marking element while firmly holding the marking tool at a desired location to mark positions. The marking of the positions is accomplished by dispensing the marking material on the body cavity tissues, which helps to facilitate a placement of sutures by the surgeon.

In yet a further embodiment, a marking tool for marking a location for implanting an artificial device by a surgeon is disclosed. The tool includes a button, a cylindrical handle, an actuator, a wire guide arrangement having a plurality of stainless steel tubes, a wire retaining plug mounted within the handle and positioned on top of a spring within the handle, a plurality of flexible wires press fitted within the wire retaining plug and positioned within the stainless steel tubes of the wire guide arrangement, and a prosthetic template connected to the handle. The handle has a central axis, a top end and a bottom end, and a bore extending through the handle around the central axis. The actuator rod is positioned within the bore of the handle and retained within the handle at a fixed location to engage the button when the button is threadably engaged with the handle. The actuator rod is retained in the handle by utilizing a pin.

The stainless steel tubes are partly mounted inside the handle and partly protruding outside the handle. A wire guide is engaged on the bottom end of the handle to seal the bore and to remain in contact with a spring positioned within the handle. The plurality of flexible wires are positioned within each respective stainless steel tube. Two or more wires are selected depending on the application.

The prosthetic template, in one embodiment, has a generally cylindrical section. The prosthetic template of the marking tool further includes a plurality of holes to accommodate the stainless steel tubes protruding out of the wire guide arrangement. The plurality of the stainless steel tubes are connected to the handle around the central axis by utilizing a support plate mounted within the prosthetic template and a support pin connected to the support plate and to the wire guide. The support pin provides additional support to the prosthetic template around the central axis of the handle.

In another embodiment of the invention, the prosthetic template includes a plurality of commissure portions. The plurality of commissure portions extends upward from the annular base corresponding to a prosthetic valve.

### BRIEF DESCRIPTION OF THE DRAWINGS

The invention is best understood from the following description when read in conjunction with the accompanying drawings. Included are the following figures:

FIG. 1 is a sectional view through the left half of a human heart showing a systolic phase of left ventricular contraction;

FIG. 2 is a sectional view through the left half of a human heart showing a diastolic phase of left ventricular expansion;

FIG. 3 is a perspective view of a prosthetic heart valve;

FIG. 4a is a perspective view of an embodiment of a suture template of the present invention;

FIG. 4b is a top view of the suture template of FIG. 4a;

FIG. 4c is a sectional view taken along line 4-4 of FIG. 4b;

FIG. 4d is a perspective view of an alternative embodiment of a suture template of the present invention.

FIG. 5 is a schematic view showing the placement of the suture template of FIG. 4a in the aorta of a patient;

FIG. 6 is a perspective view of a marking tool for facilitating a heart valve replacement;

FIG. 7 is an exploded perspective view of the marking tool of FIG. 6;

FIG. 8 is a perspective view of a prosthetic template of the marking tool of FIG. 6;

FIG. 9 is a partial cut-away view further showing an assembly of the marking tool of FIG. 6; and

FIG. 10 is a partial cut-away view of the marking tool of FIG. 6.

### DESCRIPTION OF EMBODIMENTS OF THE INVENTION

The present invention relates to a suture template for assisting surgeons in marking a location and, more specifically, to a suture template for facilitating a prosthetic heart valve replacement.

To better illustrate the advantages of the suture template of the present invention, a brief understanding of the heart and of a prosthetic heart valve is helpful. To assist in properly implanting the prosthetic heart valve, the suture template of the present invention may be employed.

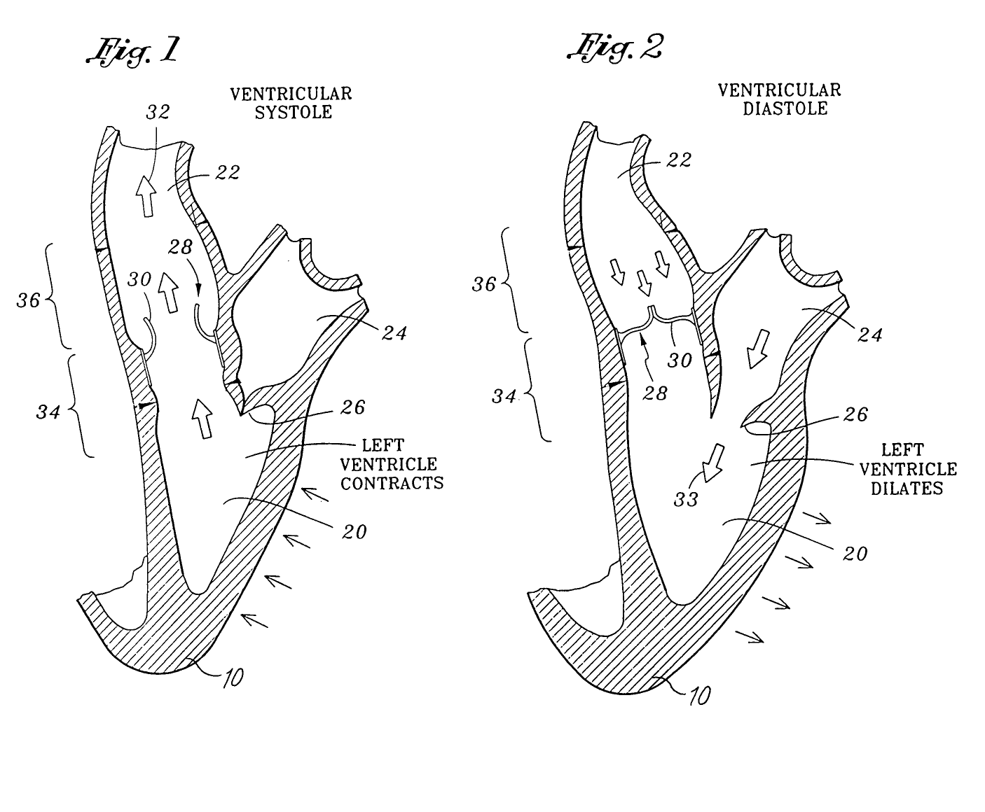

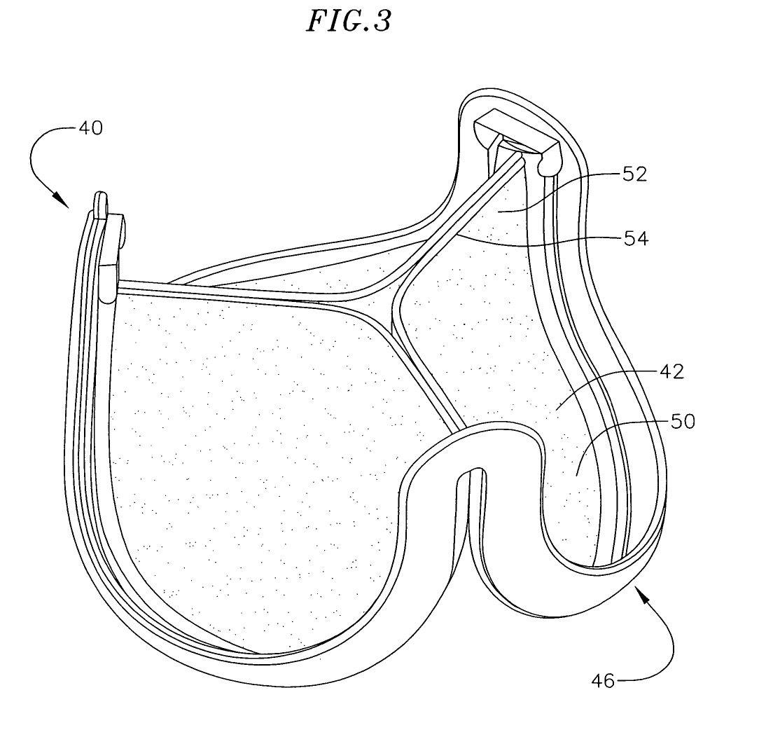

FIGS. 1 and 2 illustrate an understanding of the movement of the annulus of the aorta of the heart 10. In this regard, FIGS. 1 and 2 illustrate the two phases of left ventricular function; systole and diastole. Systole refers to the pumping phase of the left ventricle, while diastole refers to the resting or filling phase. FIGS. 1 and 2 illustrate in cross section the left chamber of the heart with the left ventricle 20 at the bottom, and the ascending aorta 22 and left atrium 24 diverging upward from the ventricle to the left and right, respectively.

FIG. 1 illustrates systole with the left ventricle 20 contracting, while FIG. 2 illustrates diastole with the left ventricle dilating. The aortic valve 28 is schematically illustrated here as having leaflets 30. A natural aortic valve typically has three leaflets, and a vertically oriented flow axis, wherein the leaflets are usually evenly distributed circumferentially about 120° apart. In this regard it will be understood that the cross-sections shown are not taken in a single plane, but instead are taken along two planes angled apart 120° with respect to one another and meeting at the midpoint of the aorta 22. Contraction of the ventricle 20 causes the mitral valve 26 to close and the aortic valve 28 to open, and ejects blood through the ascending aorta 22 to the body's circulatory system, as indicated in FIG. 1 by the arrows 32. Dilation of the ventricle 20 causes the aortic valve 28 to close and the mitral valve 26 to open, and draws blood into the ventricle from the left atrium 24, as indicated in FIG. 2 by the arrows 33.

The walls of the left chamber of the heart around the aortic valve can be generally termed the annulus region 34 and the sinus region 36. The annulus region 34 generally defines an orifice that is the narrowest portion between the ventricle 20 and the ascending aorta 22, and which is composed of generally fibrous tissue. The sinus region 36 is that area just downstream from the annulus region 34 and includes somewhat elastic, less fibrous tissue. Specifically, the sinus region 36 typically includes three identifiable, generally concave sinuses (formally known as Sinuses of Valsalva) in the aortic wall intermediate the upstanding commissures of the valve 28. The sinuses are relatively elastic and are constrained by the intermediate, more fibrous commissures of the aortic annulus. Those of skill in the art will understand that the annulus region 34 and sinus region 36 are not discretely separated into either fibrous or elastic tissue, as the fibrous commissures of the annulus extend into the sinus region 36. In addition, it will be appreciated that the valve may have only two leaflets or more than three leaflets, and that the leaflets whether two, three or more than three leaflets, may not be evenly distributed circumferentially. The suture template of the present invention is intended to be used in these less common situations also.

Each leaflet of the aortic valve has a lower cusp portion. The highest point of attachment of the leaflets to the aortic wall is called the commissure. Each commissure is connected between adjacent cusp portions and is generally axially aligned along the aortic wall. The triangular space below the commissure is called the interleaflet triangle. The tissues in this triangular space have much less dense connective tissue and are pliable and flexible. The three sinuses are located in the most proximal portion of the aorta, just above the cusps of the leaflets of the natural aortic valve. The sinuses correspond to the individual cusps of the aortic valve.

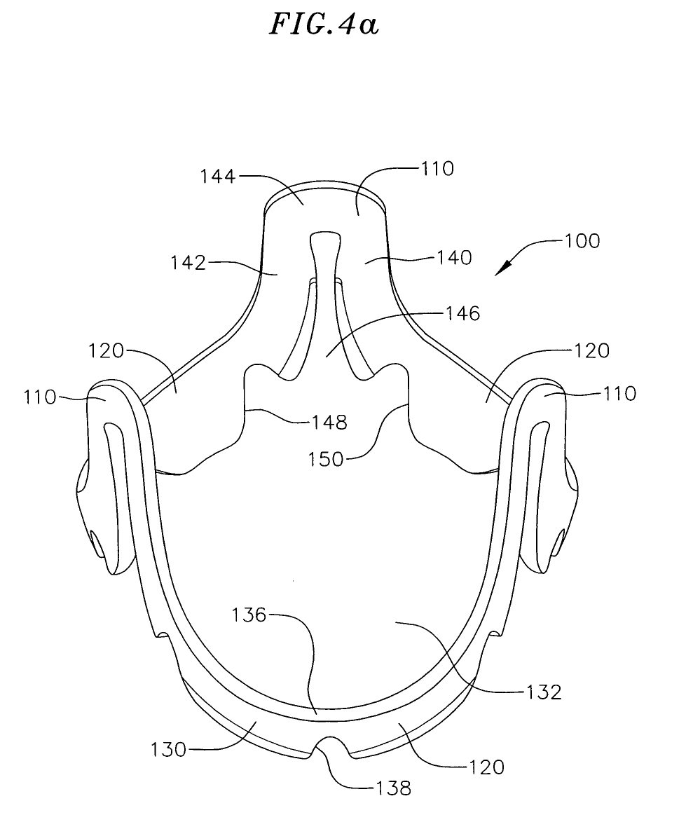

With reference to FIG. 3, a relatively new style of prosthetic heart valve 40 includes a trifoliate valve with three leaflets 42. Although three leaflets are described, and mimic the natural aortic valve, the principles of the present invention can be applied to the construction of a prosthetic valve with two or more leaflets, depending on the need.

The leaflets 42 each include an arcuate lower cusp edge 50 terminating in upstanding commissure regions 52. Each leaflet 42 includes a coapting or free edge 54 opposite the cusp edge 50. The cusp edges 50 and commissure regions 52 are secured around the periphery of the valve, with the free edges 54 permitted to meet or “coapt” in the middle. A stent assembly 46 also includes three cusps separated by three upstanding commissures. Further details regarding the structure of the heart and implanting of a prosthetic heart valve of the style illustrated in FIG. 3 is described in U.S. application Ser. No. 09/847,930, filed May 3, 2001, incorporated herein by reference. It will also be appreciated by those skilled in the art that the suture template of the present invention and its method of use may be adapted for use in any of the valves of the heart and may be used with other types of prosthetic heart valves.

Implanting the more traditional prosthetic aortic heart valves typically involves excising the natural leaflets and attaching the prosthetic heart valve along the relatively planar fibrous annulus. Implanting a valve such as that illustrated in FIG. 3 in which the sewing area more closely follows the undulating path along the leaflets'cusps and commissures is more complicated. This valve is designed to be more flexible and thus the sewing ring undulates from cusp to commissure and thus requires better sewing guidance.

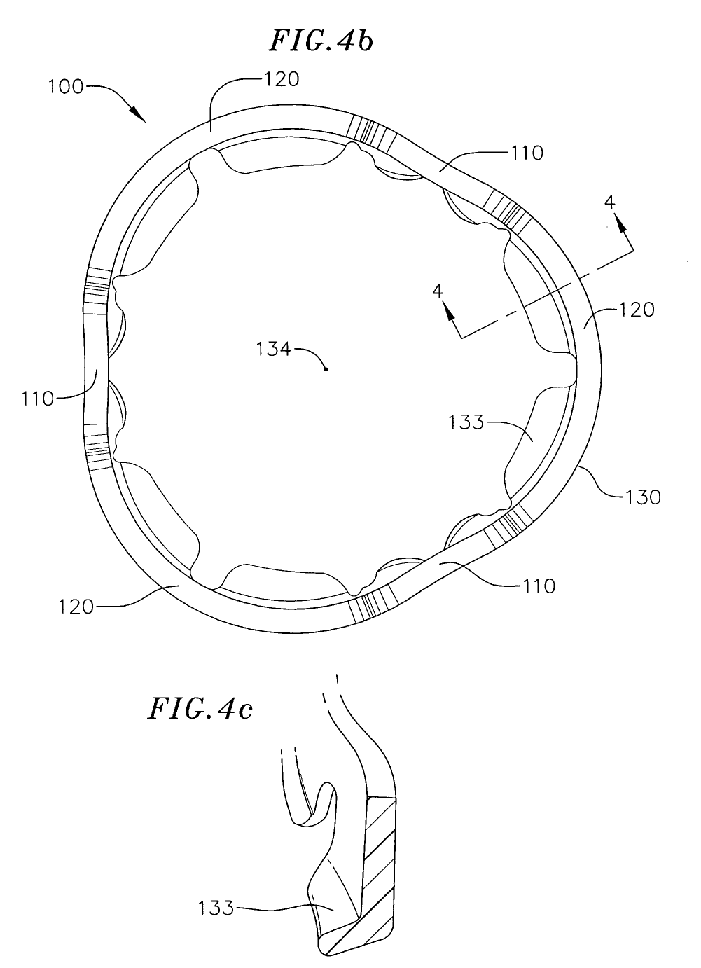

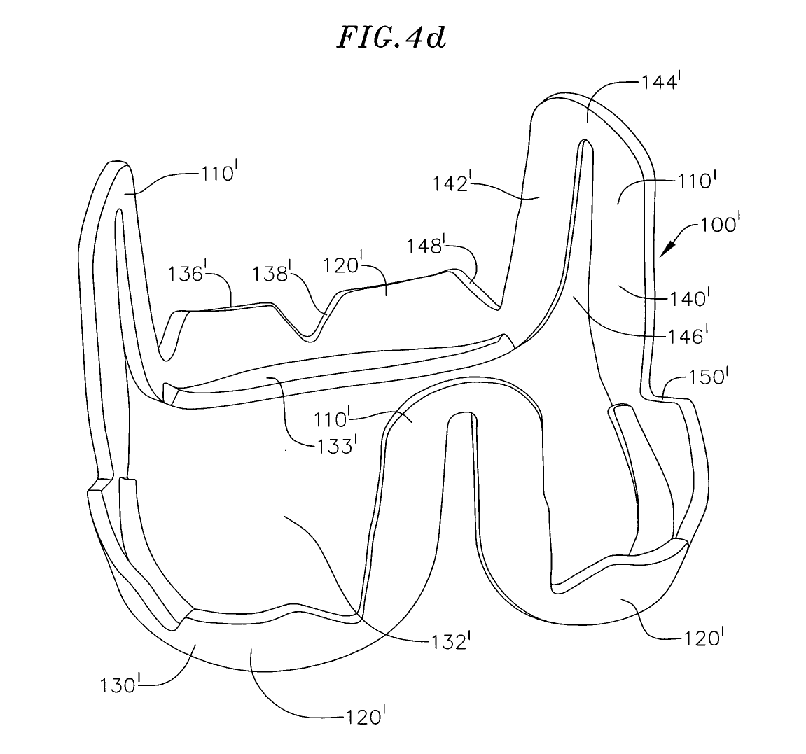

To assist in properly locating such a prosthetic heart valve, the suture template of the present invention may be employed. With reference to FIGS. 4a-4c, a suture template 100 for an aortic valve includes three commissure portions 110 alternating with three cusp portions 120. The commissure and cusp portions form an annulus defining an opening 132 therethrough having a central axis 134. The template 100 has a circumference that is sized to fit the annulus region of the aorta.

Each cusp portion 120 preferably includes a convexly curved outer surface 130 that faces radially outward, a radially inwardly extending ledge 133 to facilitate positioning of the template within the aorta and a concavely curved upper surface 136 that correlates to the bottom margin of a cusp of a leaflet of the prosthetic valve. Centrally located on each cusp are one or more downwardly opening notches 138 size to locate and capture a suture. The notches also extend through the ledge. A tab (not shown) may also be provided on an inner wall of the template to assist in placement.

Each commissure portion 110 includes a pair of upstanding arms 140, 142 that come together at the top forming a rounded tip 144. Each arm of the commissure portion extends from a respective adjacent cusp portion. The arms for each commissure portion are spaced apart at the bottom to define an elongated notch 146 that is open at the bottom and extends up to the underside of the rounded tip. The elongated notch is sized to locate and capture a suture at a location adjacent the rounded tip. Preferably, the elongated notch extends up from the base to a location above the lowermost point of the concavely curved upper surfaces 136 of the adjacent cusp portions 120. The elongated notch permits the template to fit better in the annulus around remnants of the excised leaflets and provides flexibility to the template.

Additional notches may be placed in the template to locate and capture additional sutures. For example, additional downwardly extending notches may be placed at the junctures between ends of the cusps portions and respective lower ends of the arms of the commissure portions, such as shown at 148, 150 of FIG. 4a.

FIGS. 4a-4c depict one embodiment of a template design. It will be understood by those skilled in the art, however, that several other configurations of the template are suitable for use in locating a prosthetic heart valve, including templates having different numbers of commissure and cusp portions, templates with different types of ledges or without ledges and templates with different types of surface configurations. In addition, the numbers of notches and notch placement can vary. For example, while notches 148,150 are illustrated extending downward, another embodiment would have the notches extend upward. The design of the suture template 100 is illustrated for the aortic position with three leaflets, but can be designed to the type of prosthetic heart valve to be implanted (e.g. mitral valve, pulmonary valve, tricuspid valve).

In one embodiment, the suture template is made out of a flexible material. In an exemplary embodiment, the material utilized in the suture template does not chip and is easy to cut. Suitable materials include, for example, polypropylene, polycarbonate, polystyrene or polyurethane and may be radio opaque to assist in locating the template by X ray. In another embodiment, the suture template is molded of silicone rubber.

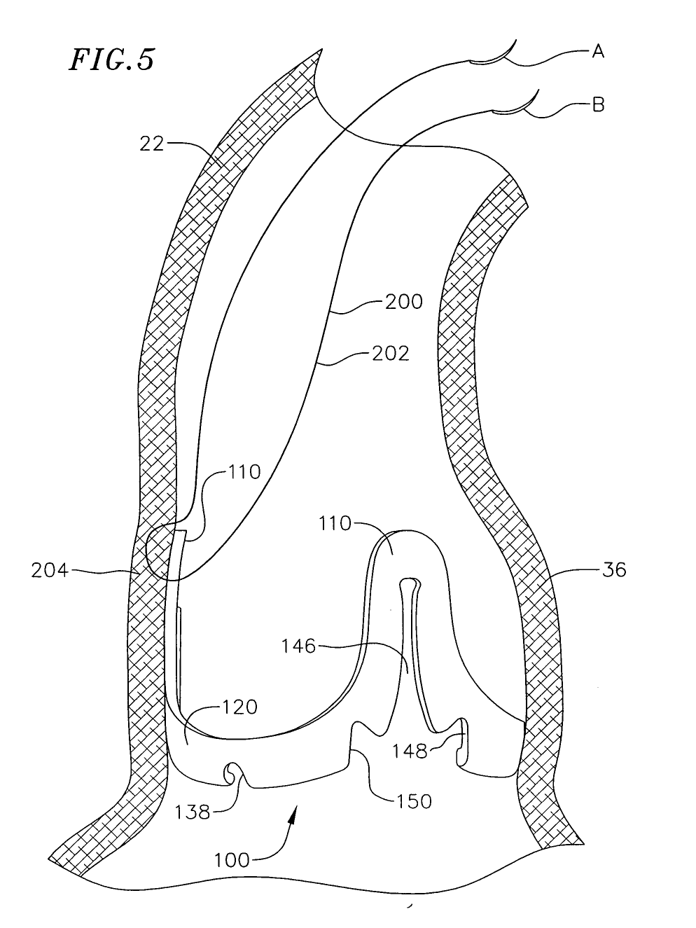

The use of the suture template 100 in aortic heart valve replacement is described below in connection with FIG. 5. A prosthetic aortic valve of the type illustrated in FIG. 3 is typically implanted on the wall of the left ventricular outflow tract mostly above the anatomic ventriculoaortic junction. The lowest point of the semilunar point of attachment of the prosthetic valve may be on the ventricular side of the junction. After the patient has been anesthetized, and the chest is opened, the top part of the heart and the aorta are visible. The patient is then placed on a heart-lung bypass machine. The cardiac surgeon then makes an incision in the aorta to gain access to the natural valve by cutting the aorta radially just above the natural valve that is to be replaced. Sutures may be generally placed at the commissures (the position on the aortic wall where two cusps meet) to hold the root open and give the surgeon easy access to the working area. Once the valve is exposed, the surgeon inspects the valve and the aortic root surrounding it to determine the extent of disease. If the disease is limited to the natural valve cusps, then the natural valve cusps are cut out. After the diseased cusps have been removed and the surgeon is satisfied that the existing root tissue is healthy, a valve sizing tool, such as described in U.S. Pat. No. 6,350,281 B1 incorporated by reference herein, may be used to determine the optimal replacement valve size.

One type of the sizer that may be utilized is disclosed in PCT International Publication Number WO 00/64382, entitled “Aortic Heart Valve Prosthesis Sizer and Marker”, which is also incorporated herein by reference. The sizer described includes a prosthesis template and a handle extending from the prosthesis template. The prosthesis template includes a generally cylindrical section and a plurality of posts along the outflow edge extending upwardly from the generally cylindrical section around the circumference of the cylindrical section. The sizer system can include a plurality of sizing elements with prosthesis templates having different diameters.

It is common practice to use the largest valve that will fit to minimize the restriction of blood flow between the valve and the annulus wall. Once the size of the prosthesis has been determined it may be “dry fitted” to ensure that the valve geometry is compatible with the patient's annulus and aortic root. Generally, the prosthetic valve is attached to a holder for ease of handling and implantation. Various types of holders are described in a U.S. application Ser. No. 09/847,930, filed May 3, 2001, entitled FLEXIBLE HEART VALVE, mentioned earlier.

Based on the size of the prosthetic valve to be implanted, the surgeon selects a suture template 100 and places it in the aorta 22, e.g. by holding a tab or a commissure portion, using e.g., forceps. The template is located such that the cusp portions 120 are aligned with the sinuses of the sinus region 36 and the commissure portions 110 are located between respective adjacent sinuses. The cusp portions are located such that the coronary artery located in two of the sinuses will remain open and unblocked to the flow of blood once the prosthetic valve is sewn in place.

Once the template 100 is in place, each notch of the template serves as a guide for receiving an individual suture 200. Each suture typically has a thread 202 and two needles, needle A and a needle B, attached at each end of the thread. In one method, the surgeon starts with a commissure portion 110 and runs the needle A of the suture 200 from inside the template through the notch 146 into the wall 204 of the aorta 22 and out of the aorta wall and back into the cavity of the aorta. The surgeon takes the needle A and the needle B and inserts them into a suture organizer, such as described in U.S. Pat. No. 4,185,636 issued on Jan. 29, 1980 to Gabbay et al. to organize various sutures. Other equivalent suture organizers available may also be utilized. The suture organizer provides an orderly and controlled arrangement of the sutures.

The surgeon then takes additional sutures and runs them individually through the two other commissure portion notches 146 and takes the needles A's and B's of each respective suture and organizes them into the suture organizer. Next, the surgeon runs new sutures through the notches 138 in the cusp portions and the notches 148, 150 at the juncture between the cusp portions and the commissure portions and organizes the needles A's and B's of each suture into the suture organizer. In another method, the surgeon may simply use the notch that is most conveniently accessible to place initial sutures rather than placing sutures initially through the commissure portion notches 110.

After the template 100 is sutured into position, the surgeon takes each needle of the suture 200 and threads it through the sewing ring of the prosthetic valve at a location corresponding to the sutures' location in the patient. At this stage, each needle A is retained in the organizer and each needle B is threaded through the sewing ring of the prosthetic valve. The surgeon repeats this process for all of the remaining sutures ensuring that needles A of all the sutures are in the organizer and needles B of all the sutures are in the prosthetic valve.

After approximately 8-12 sutures have been positioned in the aortic wall utilizing the notches of the suture template 100 and sewn through the sewing ring on the prosthetic valve, the surgeon extracts the suture template 100 from the aorta. As few as three sutures is also possible. Using the notches on the template, the template may be removed by carefully pulling it in an appropriate direction such that the sutures fall away from the notches. In addition, or alternatively, the surgeon cuts the suture template 100 in one or more places to facilitate removal of the suture template 100 from the aorta. The pieces are then removed from the patient.

Once the suture template 100 is removed, the surgeon pushes the prosthetic valve along the sutures to its proper location within the aorta and uniformly tightens the knots of each suture around the sewing ring of the prosthetic valve. Tightening of the knots secures the prosthetic valve in the proper location. If desired, the prosthetic valve may be attached to a holder, such as described in U.S. application Ser. No. 09/847,930, filed May 3, 2001, to facilitate placement of the prosthesis at the proper location.

All the sutures, once uniformly tightened, ensure the proper implantation of the prosthetic valve. Pledgets may be used to reinforce the annulus, minimizing tearing of the tissue and the failure of sutures. After the sutures are tightened, the surgeon inspects the work to make sure that blood does not leak around the valve. The surgeon also inspects that the position of the prosthetic valve does not block the blood flow to the coronary arteries and the blood flow out of the heart. After the inspection, the extraneous lengths of sutures are cut off. Once the surgeon is satisfied that the valve is positioned correctly, the aorta is sutured back together. The heart is checked for any blood leakage, air bubbles are eliminated from the heart and the patient is removed from heart-lung bypass, closed, and sent to recovery.

In the above described method, the sutures were secured to the sewing ring of the prosthetic valve prior to removing the template. It will be appreciated, however, that the template may be removed prior to securing the sutures to the prosthesis. In another alternative, after the sutures have been located in the annulus region through use of the template, the template can be removed and the prosthesis sewn directly to the annulus using the sutures.

With reference to FIG. 4d, an alternative embodiment of a template design has notches extending upward from the cusp portions. In particular, a suture template 100′ for an aortic valve includes three commissure portions 110′ alternating with three cusp portions 120′. The commissure and cusp portions form an annulus defining an opening 132′ therethrough having a central axis. The template 100′ has a circumference that is sized to fit the annulus region of the aorta.

Each cusp portion 120′ includes a convexly curved outer surface 130′ that faces radially outward, a radially inwardly extending ledge 133′ to facilitate positioning of the template within the aorta and a concavely curved upper surface 136′ that correlates to the bottom margin of a cusp of a leaflet of the prosthetic valve. Centrally located on each cusp is an upwardly opening notch 138′ that is sized to locate a suture. A tab (not shown) may also be provided on an inner wall of the template to assist in placement.

Each commissure portion 110′ includes a pair of upstanding arms 140′, 142′ that come together at the top forming a rounded tip 144′. Each arm of the commissure portion extends from a respective adjacent cusp portion. The arms for each commissure portion are spaced apart at the bottom to define an elongated notch 146′ that is open at the bottom and extends up to the underside of the rounded tip. The elongated notch is sized to locate and capture a suture at a location adjacent the rounded tip. Preferably, the elongated notch extends up from the base at least to a location above the lowermost point of the concavely curved upper surfaces 136′ of the adjacent cusp portions 120′. The elongated notch permits the template to fit better in the annulus around remnants of the excised leaflets and provides flexibility to the template.

Additional notches may be placed in the template to locate additional sutures. For example, additional upwardly extending notches may be placed at the junctures between ends of the cusps portions and respective lower ends of the arms of the commissure portions, such as shown at 148′, 150′ of FIG. 4d.

The use of this suture template 100′ is similar to the use of the suture template of FIGS. 41a-4c. However, because several notches extend upward from the cusp portions, instead of extending downward, these notches are simply used to assist in locating the suture and do not capture the suture as in the case of the downwardly extending notches shown in FIG. 5.

After sutures are positioned in the aortic wall, using the template 100′ as a guide, the template is removed by pulling it away and/or by cutting the template in one or more places to facilitate removal.

In another alternative embodiment, once the suture template 100, 100′ is strategically positioned at the annulus of the aorta, the surgeon uses a marker to mark the aorta through the notches. Once the position within the aorta is marked for the sutures, the surgeon runs the sutures through the marked positions and implants the prosthetic valve. In this embodiment, the suture template 100, 100′ is utilized simply as a guide to mark the positions for sutures rather than running the sutures through the notches as described earlier.

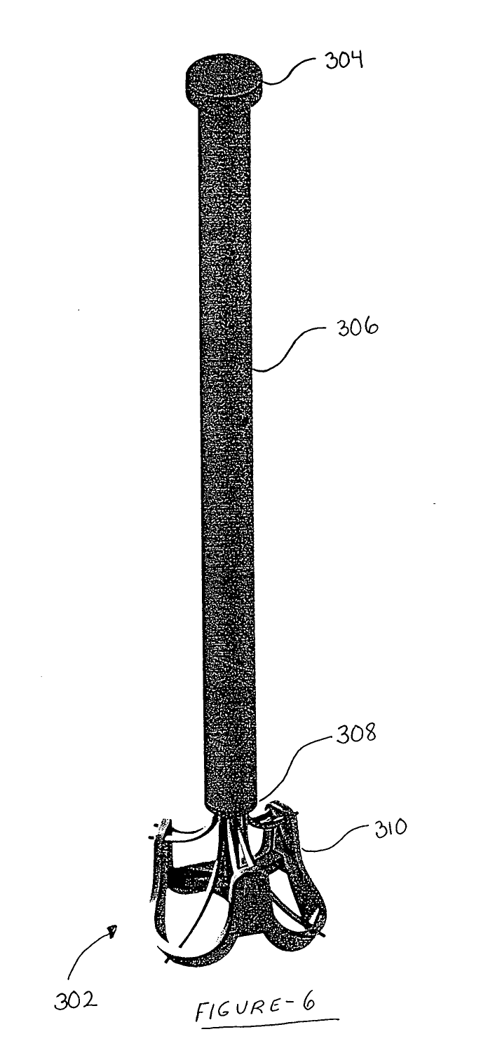

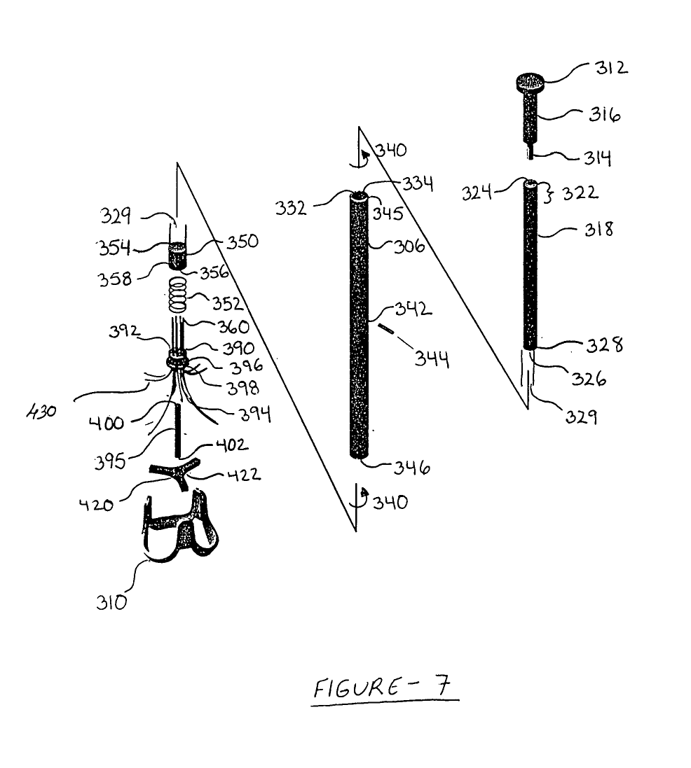

In yet another embodiment illustrated in FIG. 6, a marking tool 302,. is used for facilitating the replacement of the aortic valve. However, this is merely illustrative inasmuch as the features of this invention are equally applicable to marking positions of other heart valve replacements.

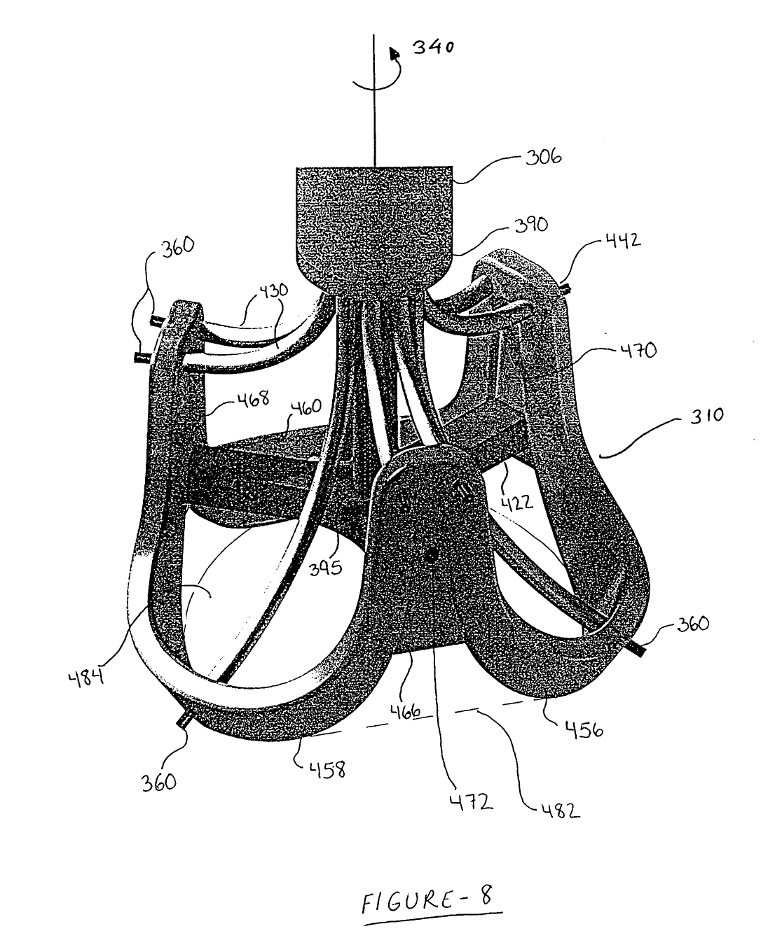

With reference to FIGS. 6 and 7, the marking tool 302 includes a button 304, a handle 306, and a wire guide arrangement 308 connected to a prosthetic template 310. The marking tool 302 is utilized by inserting the marking tool 302 within the aorta, positioning the marking tool 302 where the prosthetic device is to be implanted, and pressing the button 304 of the marking tool 302 while firmly holding the marking tool 302 at a desired location to mark positions by a marking material to facilitate the placement of sutures by the surgeon.

Referring to FIG. 7, the button 304 includes a flat head 312 at a first end, a threaded post 314 at a second end, and a middle cylindrical portion 316 between the head and the threaded post. The.middle cylindrical portion 316 is sized to fit within a central bore 334 of the handle 306. The post 314 at the second end is threaded to engage an actuator rod 318 when the actuator rod 318 is positioned within the handle 306.

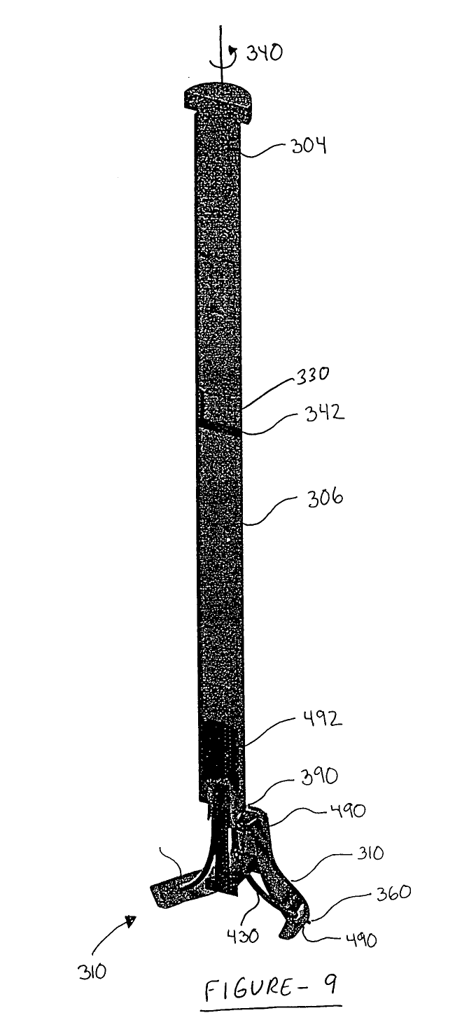

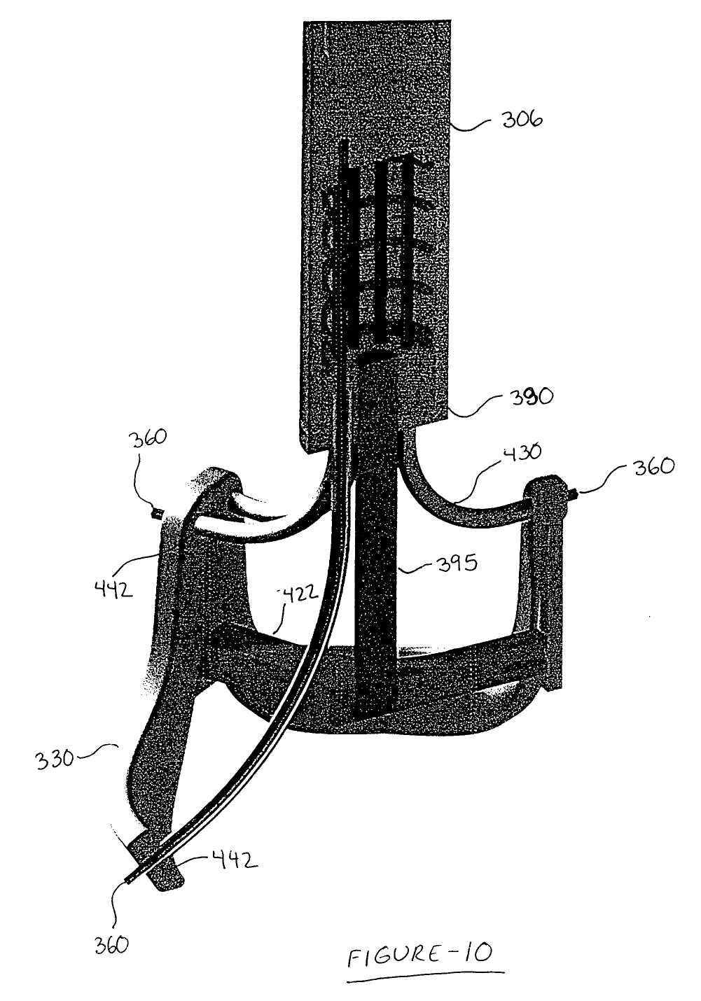

The actuator rod 318, as shown in FIG. 7, includes a threaded groove portion 322 at a top end 324 and a flat surface 326 at a second end 328. The threaded groove portion 322 includes internal threads sized to receive the threads of the post 314 of the button 304. The actuator rod 318 has an outer diameter 329 sized to fit slidably within the central bore 334 of the handle. The actuator rod 318 further includes a slot 330 (shown in FIG. 9 below) of a pre-determined length to accommodate a vertical movement of the actuator rod 318 when the button 304 is depressed to eject the nitinol wires out of the prosthetic template 310, as explained below. The details pertaining to the vertical movement is further shown and elaborated in FIGS. 9 and 10 below.

Referring again to FIGS. 6 and 7, the tubular-shaped handle 306 is formed by a cylindrical wall 332 having a central bore 334 to slidably accommodate the actuator rod 318 and a portion of the button 304. The handle 306 has an axis of symmetry 340 and a small opening 342 perpendicular to the axis of symmetry 340 to receive a pin 344. The pin 344 is positioned within the small opening 342 of the handle 306 and the slot 330 of the actuator rod 318 to facilitate the actuator movement in a vertical direction 345 when the button 304 is pressed. The handle 306 further includes a top end 345 and a bottom end 346. The top end 345 is sized to receive the actuator rod 318 as well as the button 304. The bottom end 346 is sized to receive a wire guide 390 of the wire guide arrangement 308, as explained below.

Preferably, the outer surface of the handle 306 has indentations or raised ridges to provide a gripping surface such as, for example, a knurled surface. In an alternative embodiment, the handle 306 has a finished surface. The small size of the handle 306 provides flexibility to the surgeon while positioning the marking tool 302 at the proper location within the aorta during the surgery. In one embodiment, the handle 306 has a grip (not shown). The grip can have any convenient shape for gripping. The grip can include a button or other suitable structure for implementing marking when the marking tool 302 is properly positioned within the aorta. In another embodiment, if necessary, the handle 306 can be connected to an external power supply or the like to implement marking.

The marking tool 302 further includes a wire retaining plug 350 and a spring 352. The spring 352 is provided to facilitate the vertical movement of the wire retaining plug 350, when the spring and the plug are mounted within the handle. The wire retaining plug 350 is cylindrical in shape and is sized to fit within the bore 334 of the handle 306. The wire retaining plug 350 has substantially the same diameter as the outer diameter 329 of the actuator rod 318 and includes a top surface 354 and a bottom surface 356. The top surface 354 of the wire retained plug is frictionally in contact with the second end 328 of the actuator rod 318 when the wire retaining plug 350 is mounted within the handle 306. A plurality of miniature openings 358 are provided on the bottom surface 356 of the wire retaining plug to receive a plurality of nitinol wires 360. The wires 360 are press fitted into the miniature openings 358. The wire retaining plug 350 is made out of a stainless steel material. In another embodiment, the wire retaining plug 350 is made out of a plastic or other material.

The wire guide arrangement 308 includes a wire guide 390 having a plurality of holes 392 on a periphery of the wire guide to accommodate a plurality of stainless steel tubes 430 at a pre-determined location. The wire guide 390 has a central aperture 394, which is substantially concentric with the opening 334 and is sized to receive a support pin 395. The wire guide 390 further includes a cylindrical first portion 396 connected to an annular flange 398. The cylindrical first portion 396 is sized to frictionally engage the central bore 334 of the handle 306. The annular flange 398 has an increased diameter than the first portion 396.

In this embodiment, the annular flange 398 has an outside diameter, which is substantially equal to the outside diameter of the handle 306. The spring 352 is positioned to provide the vertical movement to the actuator rod 318 when the button 304 is depressed. As discussed earlier, the button 304, when pressed, pushes the actuator rod 318 in a downward direction, which in turn, pushes the plug 350 in the same downward direction to eject the nitinol wires 360 out of the prosthetic template 310.

The plurality of holes 392 are evenly distributed along the periphery on the wire guide 390 in a pre-determined arrangement to receive a plurality of steel tubes 430. The predetermined arrangement of the holes 392 ensures that the steel tubes 430, when press fitted in the holes 392, provide the necessary structural support to the prosthetic template 310. Each steel tube is guided and press fitted through each hole of the wire guide 390. The stainless steel tubes 430 (shown in FIG. 8) are partly mounted inside the handle 306 to guide the nitinol wires 360 that are press fitted into the wire retaining plug 350 and partly protruding outside the handle 306 to connect to the prosthetic template 310. The entire wire guide arrangement 308 is then press fitted into the central bore 334 of the handle and is frictionally in contact with the bottom end 346 of the handle. In one embodiment, the wire guide 390 is formed from a plastic material such as, polypropylene, polycarbonate, or polystyrene.

The support pin 395, which is cylindrical in shape, is generally rigid and fabricated from the stainless steel material. The support pin 395 includes an upper end 400 and a lower end 402. The upper end 400 is fixedly attached to the wire guide 390, while the lower end 402 is fixedly attached into an opening 420 of a support plate 422. The support plate 422 is star-shaped. In another embodiment, the support plate 422 is generally triangular or any other suitable shape. During the assembly, the support plate 422 is positioned in the center of the prosthetic template 310 to provide the structural support. The details pertaining to the prosthetic template 310 are shown further in FIG. 8.

As shown in FIG. 8, three cusp portions 456, 458, 460 are connected with each other utilizing three commissure portions 466, 468, 470 to form the prosthetic template 310. In the embodiment shown, the three cusp portions 456, 458, 460 are molded together with three commissure portions 466, 468, 470 to form a single piece. The prosthetic template 310 further includes a plurality of openings 442 to firmly connect the plurality of stainless steel tubes 430 that are partly protruding out of the wire guide 390.

The prosthetic template 310 is assembled to the handle 306 utilizing the plurality of steel tubes 430, the support pin 395 and the support plate 422. The support plate 422 is mounted within the three cusp portions and is firmly connected to three commissure portions by utilizing a plurality of pins 472 to connect the ends of the support plate 422. The upper end 400 (as shown in FIG. 7) of the support pin 395 is fixedly attached to the wire guide 390 while the lower end 402 (as shown in FIG. 7) is fixedly attached into the opening 420 of the support plate 422 (as shown in FIG. 7) thereby connecting the support plate 422 to the handle 306. The support pin 395 is assembled with the wire guide 390 to provide additional support to the prosthetic template 310 around the axis of symmetry 340 of the handle 306. The template 310 when assembled with the support plate 422 and the support pin 395 provides additional structural support to the prosthetic template 310 around the axis of symmetry 340 of the handle 306. The template 310 when assembled forms an annular base 482 having an opening 484 therein around an axis of symmetry 340.

The plurality of nitinol wires 360 that are press fitted into the wire retaining plug 350, as described above, are positioned within the plurality of stainless steel tubes 430 to provide the marking function. The nitinol wires 360 are ejected out of their respective stainless steel tubes 430 to mark positions on body cavity tissues when the button 304 is firmly pressed by applying pressure on the first end 352. Once assembled, the button 304, the handle 306, the wire arrangement 308, the prosthetic template 310, and the actuator rod 318, all form a common axis of symmetry collinear with the axis of symmetry 340. In the embodiment shown, nine stainless steel tubes, the support pin 395, and the support plate 422 are utilized to assemble and firmly hold the prosthetic template 310 to the handle 306.

In another embodiment, flexible wires made out of materials instead of nitinol may be used. A plurality of flexible wires may be positioned within each respective stainless steel tube in a similar fashion as nitinol wires. The diameter of each respective stainless steel tube is selected to accommodate the number of flexible wires or nitinol wires selected. The prosthetic template 310 simulates the leaflets and commissures of the prosthetic valve. Different numbers of commissure portions, such as two, can be used for a prosthesis with different numbers of cusp portions. The number of steel tubes are selected based on the number of commissure portions of the prosthetic template 310.

FIG. 9 is a partial cut-away view further showing the assembly of the marking tool 302. As shown, the button 304 is attached to the actuator rod 318 positioned within the handle 306 by utilizing the threads located on the threaded post 314 at the second end of the button 304. The actuator rod 318 is positioned at a pre-determined location within the handle 306 utilizing the pin 342. The pin 342 is fixedly attached to the handle 306 and mounted within the slot 330 of the actuator rod 318 to accommodate the vertical movement of the actuator rod 318 when the button 304 is depressed by the surgeon. As shown and fully described earlier, the wire retaining plug 350, the spring 352 and the wire guide assembly 308 are positioned within the handle. Connected to the wire guide assembly 308 are the plurality of steel tubes 430, which are partially protruding from the wire guide 390 and away from the axis of symmetry 340 to connect the prosthetic template 310. The support pin 395 connected to the wire guide 390 and the support plate 422 are mounted within the prosthetic template 310, as discussed above, to provide structural support to the template 310. The prosthetic template 310 generally includes openings 442 at desired locations to accommodate the stainless steel tubes 430. The stainless steel tubes 430 are utilized to guide the nitinol wires 360 out of openings 442 and mark the tissue when the tool 302 is properly positioned. In the embodiment shown, there are nine different openings 442 on the prosthetic template 310. The number and location of the openings 442 are selected to leave desired markings within the aorta. The markings at or near the openings 442 approximately outline the position of the prosthesis against the aorta. In an alternative embodiment, the nitinol wires 360 are coated with a dry powder. This is achieved by applying ink having a very volatile thinner that dries out quickly, leaving just the dry powder at a tip of the nitinol wire. In another embodiment, a set of flexible wires are utilized instead of nitinol wires 360.

FIG. 10 is a partial cut-away view depicting the use and the operation of the marking tool 302.

The method of marking a location includes lowering the marking tool 302 within a body cavity, positioning the marking tool 302 where the prosthetic device is to be implanted within the body cavity, and pressing a button of the marking tool 302 while firmly holding the marking tool 302 at a desired location to mark positions. The marking of the positions is accomplished by dispensing the marking material in the body cavity tissues, which facilitates a placement of sutures by the surgeon.

The marking function is normally performed by the surgeon following the removal of the damaged natural heart valve and prior to implantation of the prosthetic device. The use of the marking tool improves the consistency of the replacement procedure, decreases the complexity of the attachment and reduces the implantation time. The marking function is performed by pressing the button 304 shown in FIG. 6. Pushing the button 304 in a downward direction pushes the actuator rod 318, which, in turn, pushes the wire retaining plug 350 (shown in FIG. 7) in a downward direction. The downward movement of the wire retaining plug 350 within the handle 306 ejects the nitinol wires 360 out of the steel tubes 430. As shown in FIGS. 9 and 10, the steel tubes 430 are connected to the openings 442 of the prosthetic template 310. The support plate 422 provides counter support against the downward force applied by the handle 306 thereby forcing the nitinol wires 360 out of the tubes 430 to carry the marking material or ink to mark the tissues. The nitinol wires 360 positioned within the steel tubes 430 are ejected when the actuator rod 318 mounted within the handle 306 is depressed by utilizing the button 304.

It will, of course, be understood that modifications to the present preferred embodiment will be apparent to those skilled in the art. Consequently, the scope of the present invention should not be limited by the particular embodiments discussed above, but should be defined only by the claims set forth below and equivalents thereof.

What is claimed is:

1. A suture template for facilitating implantation of a prosthetic valve in a patient, said suture template comprising:
an annular body having a plurality of commissure portions and a plurality of cusp portions, the plurality of commissure portions connected with each other utilizing the plurality of cusp portions to form an annulus having an opening therethrough;
wherein each one of said plurality of commissure portions includes a pair of upstanding arms extending from the cusp portions, the arms coming together to form a tip and the arms defining an elongated downwardly opening notch therebetween for receiving a suture.

2. The suture template of claim 1 wherein each cusp portion is provided with a notch for receiving a suture.

3. The suture template of claim 2 wherein said plurality of commissure portions extending from the cusp portions is three.

4. The suture template of claim 2 wherein the notches for the commissure portions are longer than the notches for the cusp portions.

5. The suture template of claim 2 wherein the cusp portions have concavely curved upper surfaces and wherein the notch for each commissure portion extends upwardly above lowermost points of concavely curved upper surfaces of adjacent cusp portions.

6. The suture template of claim 2 wherein the notch of each cusp portion is centrally located between two commissure portions.

7. The suture template of claim 6 wherein at least one additional notch of each cusp portion is located between each centrally located notch and the notch for each commissure portion.

8. The suture template of claim 1 wherein each cusp portion has a lower end that defines a radially inwardly directed ledge.

9. The suture template of claim 2 wherein each cusp portion has a lower end that defines a radially inwardly directed ledge and the ledge is located on each side of the notch of each cusp portion.

10. The suture template of claim 2 wherein the notch is a downwardly opening notch.

11. The suture template of claim 2 wherein the notch is an upwardly opening notch.

12. A method of attaching a prosthetic valve to a heart of a patient, said method comprising:
placing a suture template having a plurality of notches at a location of the heart that is to receive the prosthetic valve;
attaching a plurality of sutures to the location of the heart by placing the plurality of sutures through the plurality of notches of the suture template and through the location of the heart and
removing the suture template from the location of the heart; and
attaching the plurality of sutures to the prosthetic valve and fixing the prosthetic valve at said location.

13. The method of claim 12, wherein the location of the heart that is to receive the prosthetic valve is the aortic root.

14. The method of claim 12, wherein removing the suture template includes cutting the template.

15. The method of claim 12 wherein placing a plurality of sutures includes placing sutures at commissure portions of the template.

16. The method of claim 12, wherein placing a plurality of sutures includes placing sutures at commissure portions of the template and at cusp portions of the template.

17. The method of claim 16, wherein placing the plurality of sutures includes placing sutures at the commissure portions, then at the cusp portions.

18. The method of claim 12 further comprising arranging the plurality of sutures into a suture organizer before attaching the plurality of sutures to the prosthetic valve.

19. The method of claim 12 further comprising sliding the prosthetic valve along the sutures to said location after attaching the plurality of sutures to the prosthetic valve.
